(19) United States
(12) Patent Application Publication
(10) Pub. No.: US 2016/0249903 A1
(43) Pub. Date: Sep. 1, 2016

(54) Skin button used for wound healing

(71) Applicant: SHANGHAI SHILI INTERNATIONAL TRADING CO., LTD., Shanghai (CN)

(72) Inventor: Lizhong Sun, Shanghai (CN)

(21) Appl. No.: 14/761,950

(22) PCT Filed: Mar. 11, 2015
(86) PCT No.: PCT/CN2015/074047

§ 371 Date: Jul. 20, 2015

(30) Foreign Application Priority Data
Apr. 3, 2014 (CN) 201410133600.5

(51) Int. Cl. A61B 17/04; A61B 17/00

(52) U.S. Cl. CPC A61B 17/04; A61B 17/0057; A61B 2017/0404; A61B 2017/0414; A61B 2017/0419; A61B 2017/0464; A61B 2017/00637; A61B 2017/00663

(57) ABSTRACT

A skin button used for wound healing is provided, which includes a one-piece formed structure with upper layer and lower layer consisting of a 1^{st} skin button and a 2^{nd} skin button. A through hole is provided in a junction of the 1^{st} skin button and the 2^{nd} skin button, which runs through the 1^{st} skin button and the 2^{nd} skin button, so as to form a duct connected the 1^{st} skin button and the 2^{nd} skin button to an outside. The 2^{nd} skin button enters into dermis via a wound, and adheres to a medial wall of dermis at the wound. The 1^{st} skin button is fixed out of epidermis at the wound, and a position of through hole on the 1^{st} skin button is sealed and isolated from the outside. This invention is capable of effectively solving various problems in the existing technologies, with simple structure and easy operation.

### CROSS REFERENCE OF RELATED APPLICATION

This is a U.S. National Stage under 35 U.S.C 371 of the International Application PCT/CN2015/074047, filed Mar. 11, 2015, which claims priority under 35 U.S.C. 119(a-d) to CN 201410133600.5, filed Apr. 3, 2014.

### BACKGROUND OF THE PRESENT INVENTION

1. Field of Invention

This invention relates to a medical field, and particularly to a skin button used for wound healing.

2. Description of Related Arts

In clinic, whether to animal or human, after performing operation to internal organs by skin incision, usually the way to deal with the wound is to suture with stitches and suture lines. But this way would exist following problems: firstly, conduct wound suture is just after the operation, thus the rejection matter caused by organic rejection reaction cannot be timely expelled from the body; secondly, the wound can not be kept under germ-free condition; thirdly, once the wound is sutured, the situation of the operated internal organ can not be observed; fourthly, avoid skin incision once more to the sutured wound if the second operation is required, and causing re-injury to the wound; fifthly, suture with manual way, this way totally depends the operator to control the density of the suture lines, once the suture lines are sparse, that may cause obvious scar after wound healing and wound infection easily.

### SUMMARY OF THE PRESENT INVENTION

For the technical problems of above existing technology, this invention provides a skin button used for wound healing, which is able to effectively solve all the problems of above existing technology, with simple structure and easy operation.

For the purposes mentioned above, this invention adopts following technical solution:

A skin button used for wound healing includes a one-piece formed structure with upper layer and lower layer comprising a 1^{st} skin button and a 2^{nd} skin button; wherein a through hole is provided in a junction of the 1^{st} skin button and the 2^{nd} skin button, which runs through both of the upper and lower end faces of the 1^{st} skin button and the 2^{nd} skin button, so as to form a duct connected the 1^{st} skin button and the 2^{nd} skin button to an outside; the 2^{nd} skin button enters into dermis via a wound, and adheres to a medial wall of the dermis at the wound; the 1 ^{st} skin button is fixed out of epidermis at the wound, and the through hole of the 1^{st} skin button is sealed and isolated from the outside by sealing element.

The 1^{st} skin button and the 2^{nd} skin button are set by vertically symmetrical way.

The 1^{st} skin button and the 2^{nd} skin button are set by bilaterally symmetrical way.

A size of the 1^{st} skin button is set in accordance with a size of the wound.

A shape of the 1^{st} skin button and the 2^{nd} skin button is regular or irregular.

The size of the 1^{st} skin button along a wound direction is larger than the size of the 2^{nd} skin button along the wound direction.

A pore is provided in the 1^{st} skin button, so that thread ends are left out of the epidermis when adopt suture lines to suture, thus capable of straining the suture lines from the outside of the epidermis.

A spacing between the 1^{st} skin button and the 2^{nd} skin button is set according to a thickness of a skin at the wound.

The 1^{st} skin button and the 2^{nd} skin button are made of skin-allergen-free material.

Side edges of the 1^{st} skin button and the 2^{nd} skin button are smooth.

A lower end face of the 2^{nd} skin button is set with a wall border which is downwardly extended from a through hole wall, and the wall border forms one-piece with the 1^{st} skin button and the 2^{nd} skin button.

Compared with the existing technology, this invention has following beneficial effects:

1. The rejection matter caused by organic rejection reaction is expelled from the body via the duct between the 1^{st} skin button and the 2^{nd} skin button;

2. The wound is kept under germ-free condition;

3. The rehabilitation situation of the operated internal organ at any moment is able to be observed, thereby taking effective measures in time;

4. The second operation is able to be performed to the organ via the through hole if required, so as to avoid skin incision once more to the sutured wound;

5. No obvious scar on skin after wound healing;

6. Beneficial related medicine and matter are able to be applied into the wound via the duct.

### BRIEF DESCRIPTION OF THE DRAWINGS

FIG. 1 is a cross-sectional view of a skin button used for wound healing according to a first embodiment provided by this invention;

FIG. 2 is a top plan view of FIG. 1;

FIG. 3 is a cross-sectional view of the skin button used for wound healing according to a second embodiment provided by this invention;

FIG. 4 is a cross-sectional view of the skin button used for wound healing according to a third embodiment provided by this invention.

### DETAILED DESCRIPTION OF THE PREFERRED EMBODIMENT

Below will describe this invention in detail combining with specific embodiment. The following embodiment will help those skilled in the art to further understand this invention, but won't limit the invention in any way. It should be noted that the ordinary skill in the art, on the premise that won't break away from the inventive concept, can also make some transformations and improvements, and all of these belong to the scope of protection of this invention.

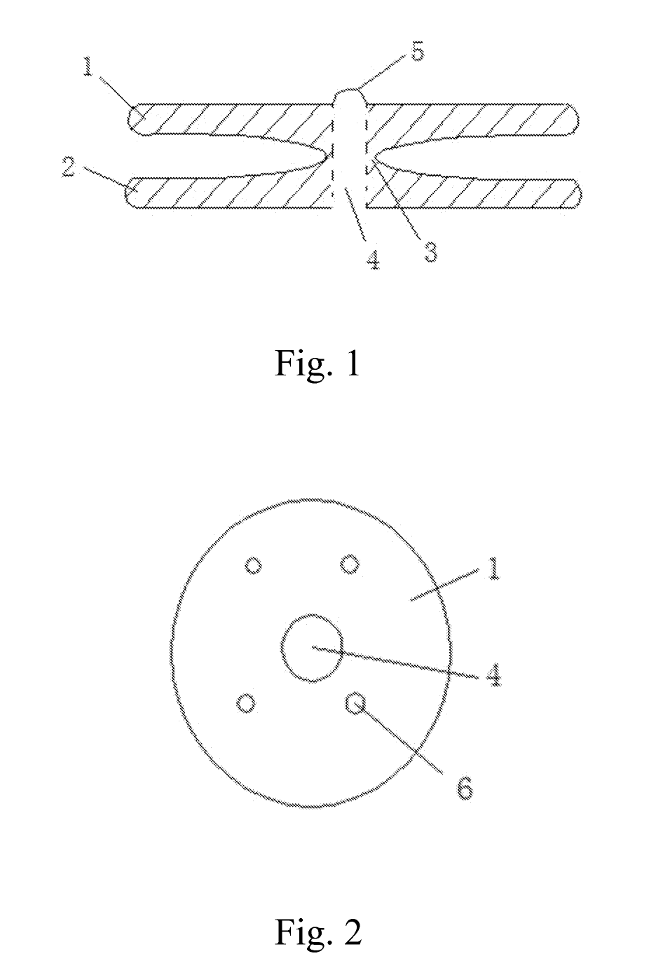

As shown in FIG. 1, a skin button used for wound healing according to a first embodiment provided by this invention is provided, which includes one-piece formed structure with upper layer and lower layer, which in the figure respectively are a 1^{st} skin button 1 located on an upper part and a 2^{nd} skin button 2 located on a lower part. A through hole 4 runs through the 1^{st} skin button 1, the 2^{nd} skin button 2 and a junction 3 therebetween, so as to form a duct connected the 1^{st} skin button 1 and the 2^{nd} skin button 2 to an outside.

While using, the 2^{nd} skin button 2 passes through a wound and enters into dermis, an inside wall of the 2^{nd} skin button 2 adheres to a medial wall of the dermis at the wound. The 1^{st} skin button 1 is fixed out of epidermis at the wound, so that a skin at the wound is clamped on a position between the 1^{st} skin button 1 and the 2^{nd} skin button 2. With a natural physiological reaction of the skin, the skin closely adheres to an inside wall of the r^{t} skin button 1 to grow for rehabilitation. The through hole of the r^{t} skin button is sealed and isolated from the outside by a sealing element 5.

Wherein, the 1^{st} skin button 1 and the 2^{nd} skin button 2 are able to be vertically symmetrical or bilaterally symmetrical, a size and a shape thereof are also able to be set flexibly according to a specific situation of the wound such as the size, shape, etc. The skin at the wound is clamped on the position between the 1^{st} skin button 1 and the 2^{nd} skin button 2, thus a spacing between the 1^{st} skin button 1 and the 2^{nd} skin button 2 is able to be set according to a thickness of the skin.

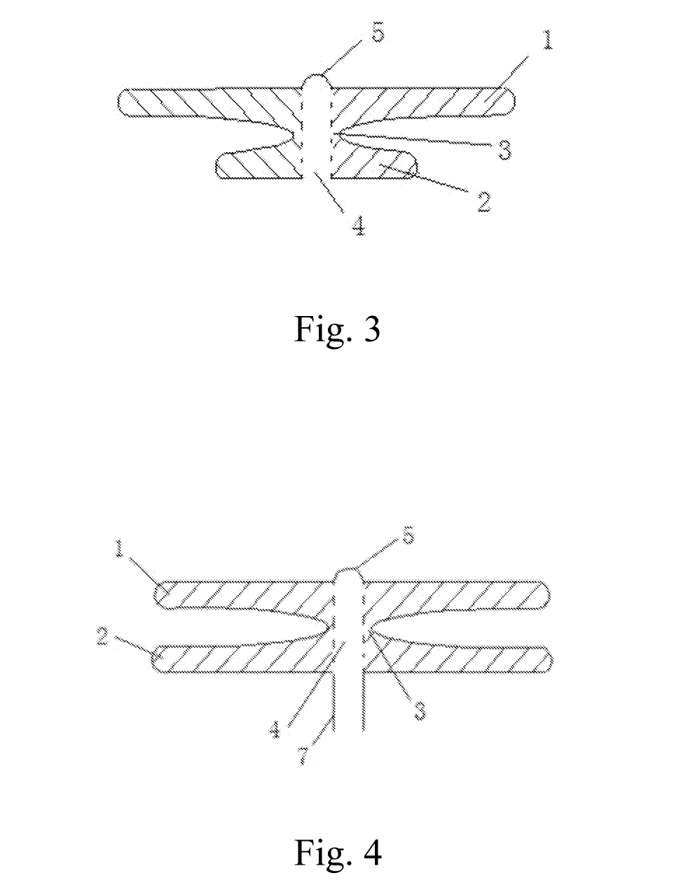

In practice, the 1^{st} skin button 1 is bare out of the epidermis, while the 2^{nd} skin button 2 is located in the skin which plays a role of fixing the 1^{st} skin button 1. Therefore, the size of the 1^{st} skin button 1 needs to coordinate to a wound length, while the size of the 2^{nd} skin button 2 only need to be set on the premise that meets a requirement of fixing the 1^{st} skin button 1, which means the size of the 1^{st} skin button 1 along a wound direction is allowable to be larger than the size of the 2^{nd} skin button 2 along the wound direction, as shown in FIG. 3, which is the skin button used for wound healing according to a second embodiment provided by this invention.

FIG. 4 shows the skin button used for wound healing according to a third embodiment provided by this invention, a wall border 7 is set at an end face of the 2^{nd} skin button which is downwardly extended from a through hole wall, and the wall border 7 is one-piece formed with the 1^{st} skin button 1 and the 2^{nd} skin button 2. When the 2^{nd} skin button 2 passes through the wound and enters into the dermis, the inside wall of the 2^{nd} skin button 2 adheres to the medial wall of the dermis. The wall border 7 stretches into the dermis space.

The 1^{st} skin button 1 and the 2^{nd} skin button 2 are made of skin-allergen-free material. For avoiding scratching skin on operation, side edges of the 1^{st} skin button and the 2^{nd} skin button are designed as curve form for smooth transition.

In addition, when wound size is quite long or not appropriate to adopt large-size skin button, the way of suturing with suture lines on the wound area where the skin button cannot cover is adopted. At this time, one or more pores 6 are provided in the 1^{st} skin button 1, as shown in FIG. 2. After well-sutured, thread ends are put through the pore 6 until outside of the epidermis, thus it is able to strain the suture lines from the outside of the epidermis.

Above describes the specific embodiment of this invention. Need to understand that this invention is not limited to above-mentioned specific implementation way, and those skilled in the art can make various transformations or modifications within the claims, that wouldn't affect the substantive content of this invention.

What is claimed is:

1. A skin button used for wound healing, including a one-piece formed structure with upper layer and lower layer including a 1^{st} skin button and a 2^{nd} skin button; wherein a through hole is provided in a junction of the 1^{st} skin button and the 2^{nd} skin button, which runs through both of upper and lower end faces of the 1^{st} skin button and the 2^{nd} skin button, so as to form a duct connected the 1^{st} skin button and the 2^{nd} skin button to an outside; the 2^{nd} skin button enters into dermis via a wound, and adheres to a medial wall of the dermis at the wound; the 1^{st} skin button is fixed out of epidermis at the wound, and the through hole of the 1^{st} skin button is sealed and isolated from the outside by sealing element.

2. The skin button used for wound healing according to claim 1, wherein the 1^{st} skin button and the 2^{nd} skin button are set by vertically symmetrical way.

3. The skin button used for wound healing according to claim 1, wherein the 1^{st} skin button and the 2^{nd} skin button are set by bilaterally symmetrical way.

4. The skin button used for wound healing according to claim 1, wherein a size of the 1^{st} skin button is set in accordance with a size of the wound.

5. The skin button used for wound healing according to claim 1, wherein a shape of the 1^{st} skin button and the 2^{nd} skin button is regular or irregular.

6. The skin button used for wound healing according to claim 1, wherein a size of the 1^{st} skin button along a wound direction is larger than a size of the 2^{nd} skin button along the wound direction.

7. The skin button used for wound healing according to claim 1, wherein a pore is provided in the 1^{st} skin button, so that thread ends are left out of the epidermis when adopt suture lines to suture, thus straining the suture lines from the outside of the epidermis.

8. The skin button used for wound healing according to claim 1, wherein a spacing between the 1^{st} skin button and the 2^{nd} skin button is set according to a thickness of the skin at the wound.

9. The skin button used for wound healing according to claim 1, wherein the 1^{st} skin button and the 2^{nd} skin button are made of skin-allergen-free material.

10. The skin button used for wound healing according to claim 1, wherein side edges of the 1^{st} skin button and the 2^{nd} skin button are smooth.

11. The skin button used for wound healing according to claim 1, wherein a lower end face of the 2^{nd} skin button is set with a wall border which is downwardly extended from a through hole wall, and the wall border forms one-piece with the 1^{st} skin button and the 2^{nd} skin button.
